(19) United States
(12) Patent Application Publication
(10) Pub. No.: US 2026/0335117 A1
(43) Pub. Date: Oct. 29, 2026

(54) CERVICAL NECK COLLAR

(71) Applicant: Thomas Jeffeson University, Philadelphia, PA (US)

(72) Inventor: Jennifer Mary Hegelein, Southampton, NJ (US)

(21) Appl. No.: 19/647,362

(22) Filed: Apr. 14, 2026

Related U.S. Application Data
(60) Provisional application No. 63/788,283, filed on Apr. 14, 2025.

(51) Int. Cl. A61F 5/055

(52) U.S. Cl. CPC A61F 5/055

(57) ABSTRACT

An adjustable cervical collar comprises a collar body comprising a base configured to rest on shoulders of a user, and a neck extending upward from the base, the neck comprising at least one connection point, a chest support comprising first and second portions hingedly connected to the collar body, and configured to secure the cervical collar about a neck of the user when connected to one another, and a chin support attachment configured to detachably connect to the connection point of the collar body. A method of fabricating a cervical neck collar is also disclosed.

### CROSS-REFERENCE TO RELATED APPLICATIONS

This application claims priority to U.S. Provisional Application No. 63/788,283, filed on Apr. 14, 2025, the contents of which are incorporated by reference herein in their entirety.

### BACKGROUND OF THE INVENTION

Cervical collars are used to provide head and neck support to patients following injury or a surgical procedure, or to patients requiring longer-term neck support. These patients may suffer from chronic pain, muscular degeneration, muscle weakness or stiffness or other chronic conditions. Cervical collars come in two major types: medical grade collars, and over the counter (OTC) collars. Medical grade collars are not designed for patients who require long-term or intermittent support since they fully immobilize the head, are bulky and uncomfortable, and require the aid of medical staff or a caregiver to don or doff. Conventional OTC collars, although meant for intermittent support, are similarly overly restrictive, fit awkwardly and are difficult for patients to don and doff independently. Patients' head and neck mobility is extremely limited and as such, patients have difficulty in performing daily activities while wearing cervical collars. Additionally, cervical collars come in few sizes and do not account for the unique and asymmetrical shapes of a patient's neck and shoulder, often causing bruising, pinching and pain when worn for long periods of time.

Thus, there is a need in the art for a custom fitted and mobility enhancing cervical collar that allows patients to easily perform their daily activities while keeping their head and neck supported.

### SUMMARY OF THE INVENTION

In one aspect, an adjustable cervical collar comprises a collar body comprising a base configured to rest on shoulders of a user, and a neck extending upward from the base, the neck comprising at least one connection point, a chest support comprising first and second portions hingedly connected to the collar body, and configured to secure the cervical collar about a neck of the user when connected to one another, and a chin support attachment configured to detachably connect to the connection point of the collar body.

In one embodiment, the chin support is configured to detachably connected to the at least one connection point via at least one pivot connection, configured to allow the chin support to rotate about a pivot axis. In one embodiment, the at least one connection point comprises left and right connection points, and the chin support is configured to detachably connect to the left and right connection points via left and right pivot connections to allow the chin support to rotate about a pivot axis. In one embodiment, the pivot axis is parallel to a transversal axis of the user. In one embodiment, the first and second side portions of the collar body comprise an actuator configured to rotate the chin support attachment through a range of motion.

In one embodiment, the cervical collar further comprises a button configured to allow rotation of the actuator about the pivot axis when pressed, and lock the chin support attachment in position relative to the pivot axis when released. In one embodiment, the at least one connection point comprises a guide structure, allowing the user to easily attach the chin support attachment by guiding the chin support attachment into place, without the need for a direct line of sight. In one embodiment, the collar further comprises a release mechanism configured such that, by pressing on a switch located near the connection point, releases the chin support attachment from the collar body.

In one embodiment, the first and second portions of the chest support are hingedly attached to one another at a chest support attachment point to allow for pivotal movement about a chest pivot axis such that the first and second portion of the chest support can each be rotated independently. In one embodiment, the chest pivot axis is parallel to the frontal plane of the user. In one embodiment, the chest support attachment point further comprises a detent that restricts the angular range of motion of the first and second portions of the chest support relative to one another about the chest pivot axis. In one embodiment, the first and second portions of the chest support are configured to be rotated to create a size-adjustable opening, through which the collar can be donned by the user.

In one embodiment, the first and second portions of the chest support are releasably attached at the chest support attachment point via a strap buckle which secures the cervical neck collar to the user. In one embodiment, the body, chest support and chin support attachment are made of a material selected from plastics, metals, any organic or nonorganic polymers, polylactic acid (PLA), thermoplastic polyurethane (TPU), or composite materials. In one embodiment, at least one of the collar body, chest support and chin support attachment is asymmetrical and sized or shaped in a way to fit specific anatomy of the user.

In one aspect, a method of fabricating a cervical neck collar comprises the steps of obtaining user measurements via a 3-dimensional scan, producing a computerized model of a cervical neck collar that conforms to the user's measurements, 3D printing a cervical neck collar components, wherein the components comprise a collar body, a shoulder support, and a chin support attachment, and assembling the components to form the cervical neck collar.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A, FIG. 1B, FIG. 1C, FIG. 1D, and FIG. 1E depict an exemplary embodiment of a cervical neck collar. FIG. 1A depicts a front perspective view of an exemplary cervical neck collar. FIG. 1B depicts a right perspective view of an exemplary cervical neck collar. FIG. 1C depicts a left perspective view of an exemplary cervical neck collar. FIG. 1D depicts a back perspective view of an exemplary cervical neck collar. FIG. 1E depicts a top perspective view of an exemplary cervical neck collar.

FIG. 2A and FIG. 2B depict an exemplary embodiment of a cervical neck collar with the chest supports rotated outwards for donning and doffing, and the chin support detached. FIG. 2A depicts a side perspective view of the cervical neck collar. FIG. 2B depicts a top perspective view of the cervical neck collar.

FIG. 3A, FIG. 3B and FIG. 3C depict an exemplary embodiment of the housing of the chin support pivot mechanism. FIG. 3A and FIG. 3B depict the inner structure of the housing. FIG. 3C depicts a magnet release switch located on the underside of the housing.

FIG. 4A and FIG. 4B depict an exemplary embodiment of a cervical neck collar. FIG. 4A depicts an embodiment of a cervical neck collar with the chest support at a neutral position. FIG. 4B depicts an embodiment of a cervical neck collar with the chest support rotated downwards to allow the user to look down.

FIG. 5A and FIG. 5B depict the steps in the donning process of the cervical neck collar.

FIG. 6A and FIG. 6B depict the cervical neck collar being worn by a user. FIG. 6A depicts the user wearing the cervical neck collar with the chin support adjusted to a neutral position. FIG. 6B depicts the user wearing the cervical neck collar with the chin support angled downwards.

FIG. 7 depicts the steps in the process of adjusting the chin support position.

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” “user,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Cervical Neck Collar

The disclosed cervical collar provides a cervical collar alternative for patients suffering from chronic neck pain or neck muscle weakness or stiffness. The disclosed cervical collar provides many advantages over conventional cervical collars by providing a customized fit to the user, a flat design to reduce pressure points, bruising and injuries, and an adjustable chin support which enhances mobility of the head and neck. The disclosed cervical collar is also configured for independent donning and doffing, facilitating its use as a long-term head and neck support device.

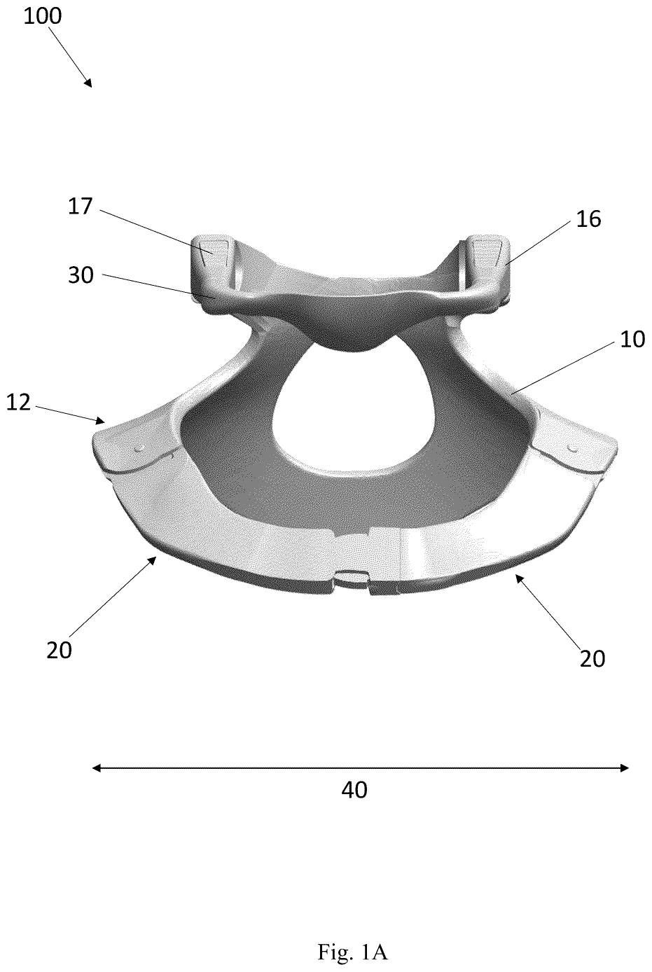

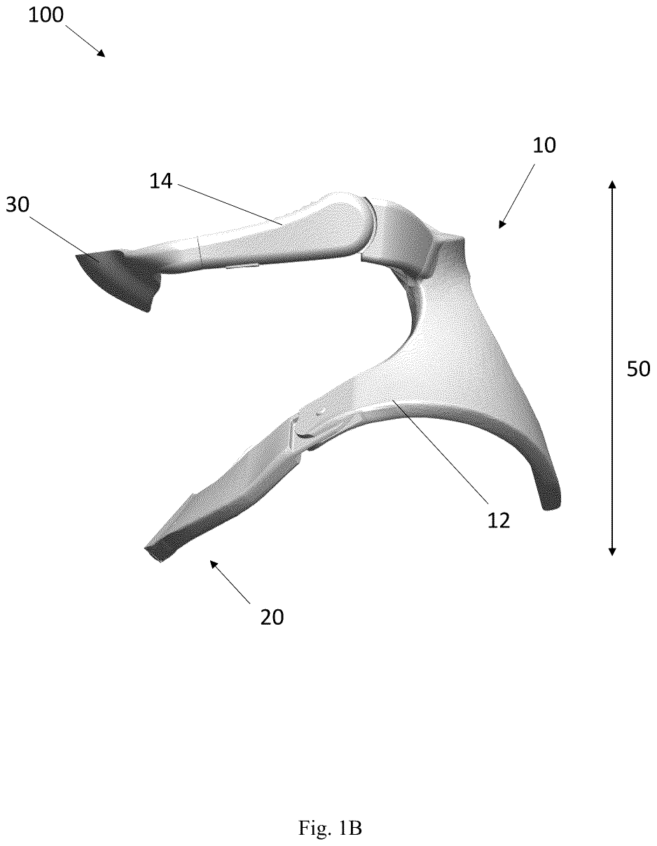

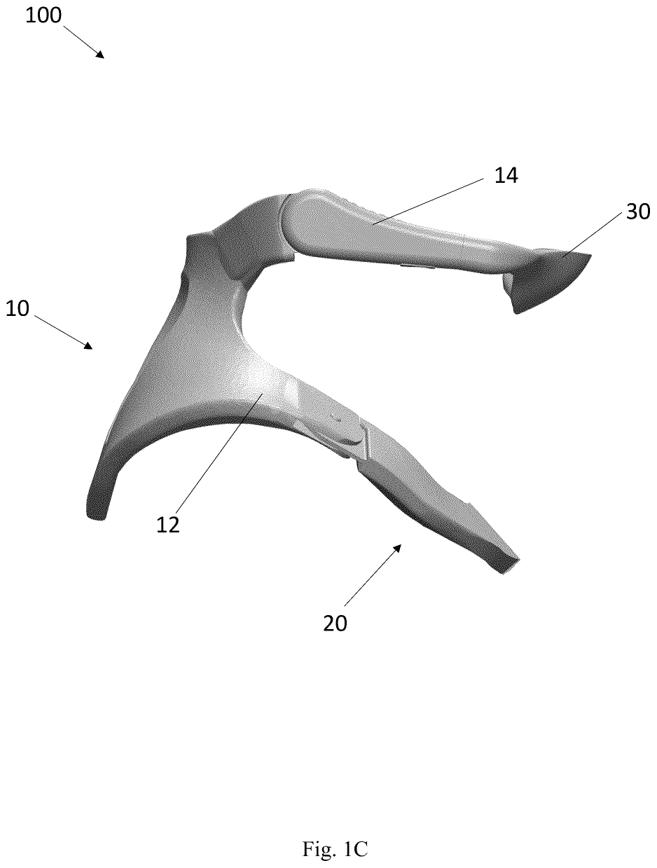

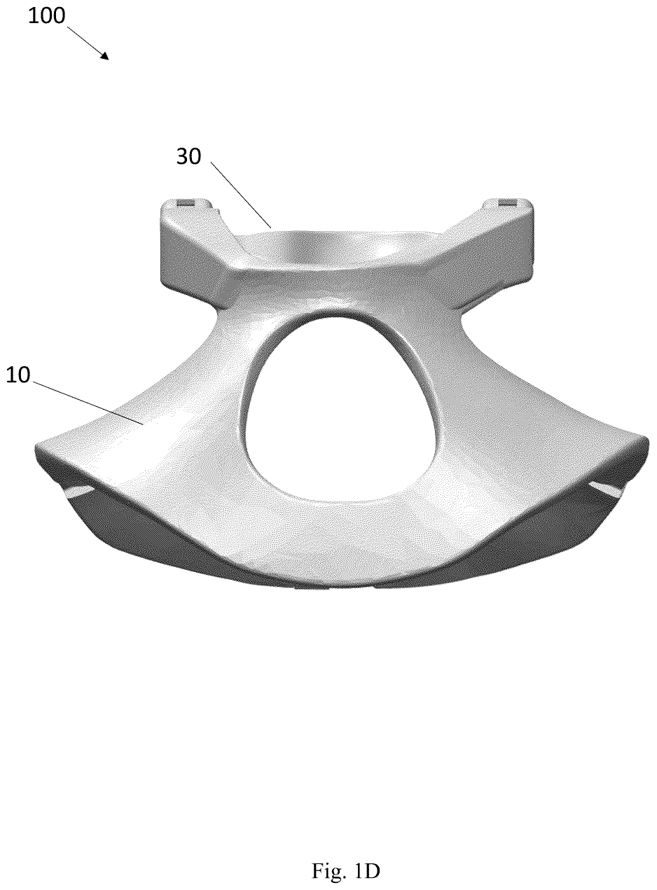

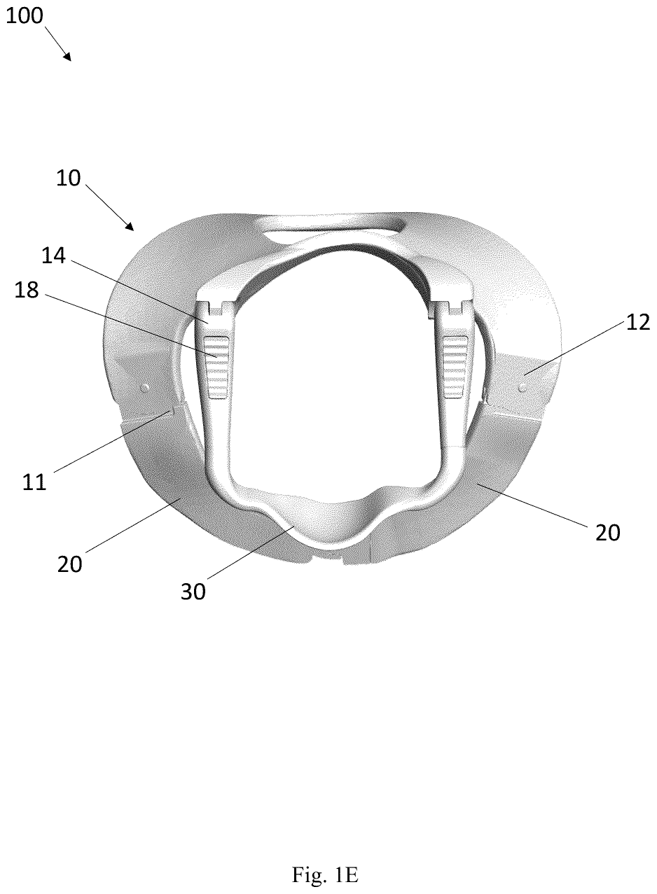

Referring now to FIG. 1A-1F, an exemplary embodiment of a cervical collar 100 is shown. Collar 100 generally includes a collar body 10, chest support 20 and a chin support attachment 30. In some embodiments, body 10, chest support 20 and chin support 30 are separate structures assembled together to form collar 100. In some embodiments, chest support 20 and chin support 30 are releasably attached to body 10.

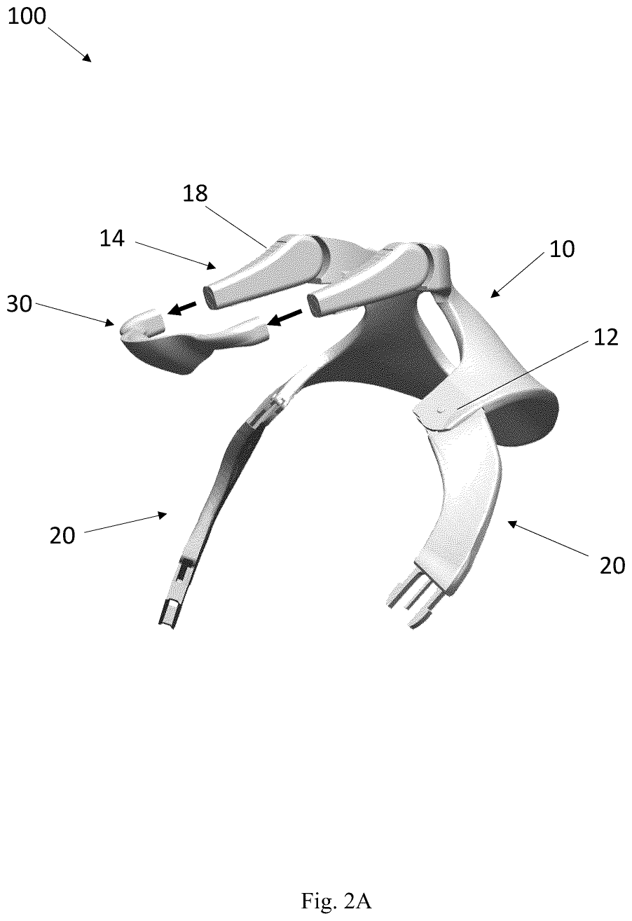

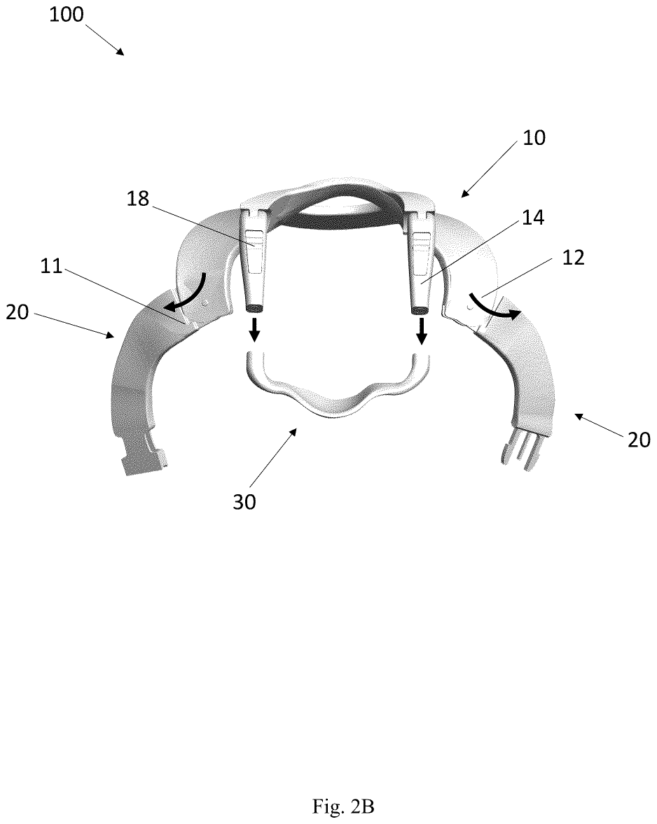

Referring now to FIGS. 2A and 2B, collar body 10 is made of a flat plane of material, with an inner surface and an outer surface. In some embodiments, body 10 may be shaped and/or sized in any way such as to conform to the user's anatomy. In some embodiments, body 10 may be an asymmetrical structure. In some embodiments, the inner surface of body 10 is configured to interface with the body of the user. In some embodiments, a cushioning layer is attached to the inner surface of body 10. In some embodiments, the outer surface of body 10 may be smooth, textured, or a combination of smooth and textured portions. In some embodiments, body 10 is shaped and sized in any way such that it lies flat against a user's skin and can be worn under clothing.

The exemplary body shown in FIG. 1A-1D includes a base configured to rest on the shoulders of the user, the base having a ventral end comprising an attachment mechanism 20, configured to secure the body about the neck of the user, and a dorsal portion having one or more protrusions extending upward to form a neck portion. Protrusions 13 extend further at a right angle such that it wraps around the jawline of the user, and further comprises a connection point, configured to secure the chin support attachment to the base.

In some embodiments, body 10 is made of a rigid material. For example, and without limitation, body 10 can be made of plastics, metals, any organic or nonorganic polymers, polylactic acid (PLA), thermoplastic polyurethane (TPU), or composite materials. In some embodiments, body 10 is made of a rigid material layered with a softer material for a cushioning effect. For example, and without limitation, rubber, nylon, silicon, foam, or gel padding may be used. In some embodiments, a cushioning layer is attached to the inner surface of body 10. In some embodiments, the cushioning layer is attached to one or more portions of the inner surface of body 10. In some embodiments, the cushioning layer is attached using any suitable method known in the art. For example, and without limitation, adhesives, hook and eye closures, or Velcro may be used.

In some embodiments, the width 40 of body 10 may be between about 20 cm and 60 cm, between about 25 cm and 55 cm, between about 30 cm and 50 cm, between about 35 cm and 45 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, or about 55 cm.

In some embodiments, the height 50 of body 10 may be between about 20 and 40 cm, between about 20 cm and 30 cm, between about 30 cm and 40 cm, about 20 cm, about 21 cm, about 22 cm, about 23 cm, about 24 cm, about 25 cm, about 26 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, or about 34 cm.

In some embodiments, the thickness of body 10 may be between about 1 and 5 mm, between about 2 and 4 mm, between about 2 and 3 mm, about 1 mm, about 1.5 mm, about 1.6 mm, about 1.7 mm, about 1.8 mm, about 1.9 mm, about 2 mm, about 3 mm, about 4 mm, or about 5 mm.

Referring now to FIG. 2A and FIG. 2B, collar 100 further comprises chest support 20. In some embodiments, chest support 20 comprises a flat strip of rigid material with an inner surface and an outer surface. In some embodiments, chest support 20 may be an asymmetrical structure. In some embodiments, the inner surface of chest support 20 is configured to interface with the upper thorax of a user. In some embodiments, chest support 20 is a continuous strip of material with an opening at a medial point. In some embodiments, chest support 20 is shaped and sized in any suitable way such that it lies flat against a user's skin and can be worn under clothing.

In a preferred embodiment, chest support 20 is comprised of a first portion and a second portion attached to a respective first and second side of body 10 at lateral sides 12. In some embodiments, the first and second portions of chest support 20 are releasably attached at a medial point to secure collar 100 to the user. In some embodiments, the two portions of chest support 20 are releasably attached using any suitable method known in the art. Examples include, but are not limited to, hook and eye closures, Velcro, ties, depressible buttons, magnets, and strap buckles. In a preferred embodiment, chest supports 20 are attached using strap buckles.

In some embodiments, and referring now to FIG. 2A and FIG. 2B, the first and second portions of chest support 20 are attached to a respective first and second side of body 10 at lateral sides 12. Chest supports 20 may be attached to body 10 using any suitable method known in the art. Exemplary attachment methods include, without limitation, adhesives, pin and groove mechanisms, magnetic connections, hinge mechanisms, pivot mechanisms and the like.

In some embodiments, the first and second portions of chest support 20 are attached to body 10 via a pivot mechanism. In some embodiments, pivot connections are located at the ends of the first and second portions of chest support 20 and a respective first and second lateral side 12 of body 10. The pivot connections allow for a rotational movement of the first and second portions of chest support 20 about a pivot axis, such that the angle subtended by body 10 and chest support 20 can be adjusted through an angular range. In some embodiments, the pivot connections comprise detents 11 located on the inner side of the pivot connections, such that the first and second portions of chest 20 are prevented from rotating beyond an angular range, thereby eliminating unnecessary movement and complications during donning and doffing. In some embodiments, the first and second portions of chest support 20 can be rotated through an angular range of between about 0° and 135°. In some embodiments, an opening is created between the first and second portions of chest support 20, the size of which can be adjusted by rotating the first and second portions of chest support 20. The opening is closed, and collar 100 is secured around the user's neck when the first and second portions of chest support 20 are attached together.

In some embodiments, chest supports 20 are made of a rigid material. For example, and without limitation, chest supports 20 can be made of any organic or nonorganic polymers, plastics, polylactic acid (PLA), thermoplastic polyurethane (TPU), or composite materials. In some embodiments, chest supports 20 are made of a layer of rigid material with an outer layer of softer material for a cushioning effect. For example, and without limitation, rubber, silicone, foam or gel padding may be used. In some embodiments, a cushioning layer is attached to the inner surface of body 10. In some embodiments, the cushioning layer is attached to one or more portions of the inner surface of body 10. In some embodiments, the cushioning layer is attached using any suitable method known in the art. For example, and without limitation, adhesives, hook and eye closures, or Velcro may be used.

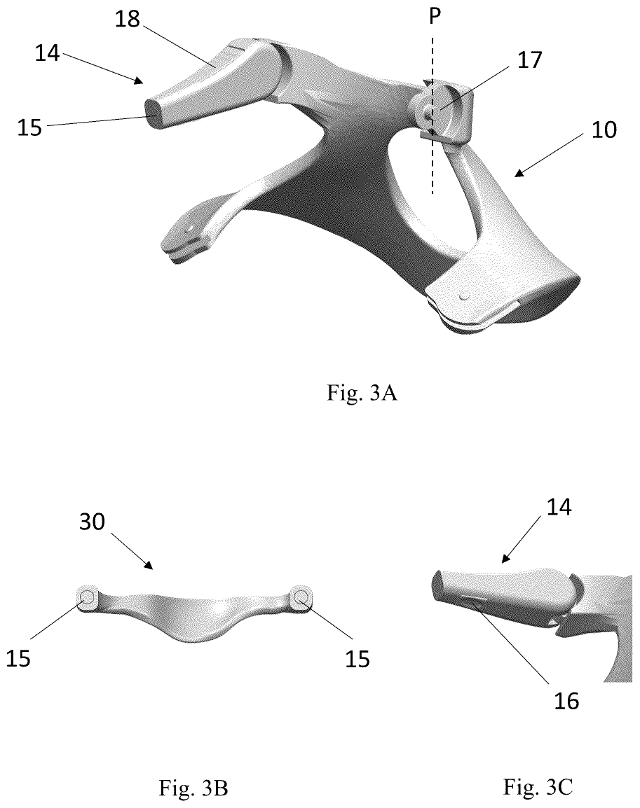

In some embodiments, and referring now to FIG. 2B and FIG. 3A, FIG. 3B, and FIG. 3C, collar 100 further comprises chin support 30. In some embodiments, chin support 30 is releasably attached to body 10. In some embodiments, chin support 30 is a flat, curved structure with an inner surface and an outer surface. In some embodiments, the inner surface of chin support 30 is adapted to conform to the shape of a user's chin.

In some embodiments, chin support 30 is made of a rigid material. For example, and without limitation, chin support 30 can be made of any organic or nonorganic polymers, plastics, polylactic acid (PLA), thermoplastic polyurethane (TPU), or composite materials. In some embodiments, chin support 30 is made of a rigid material and layered with in a softer material for a cushioning effect. For example, and without limitation, rubber, silicon, foam or gel padding may be used. In some embodiments, a cushioning layer is attached to the inner surface of body 10. In some embodiments, the cushioning layer is attached to one or more portions of the inner surface of body 10. In some embodiments, the cushioning layer is attached using any suitable method known in the art. For example, and without limitation, adhesives, hook and eye closures, or Velcro may be used.

In some embodiments, body 10 further comprises a first and second housing 14 located at a respective first and second lateral side of body 10. In some embodiments, housing 14 houses an attachment mechanism configured to interface with chin support attachment 30. Exemplary attachment mechanisms include, without limitation, a friction fit mechanism, ball and socket mechanisms, pin and groove mechanisms, magnetic connections, and the like.

In some embodiments, and referring now to FIG. 3A and FIG. 3B, chin support 30 is releasably attached to body 10 using magnetic connections 15. In some embodiments, the magnetic attachment mechanism is configured such that the ends of housings 14 each contain a magnetic female connection while the respective first and second side portions of chin support 30 each contain a male magnetic connection. When chin support 30 is brought a small distance from the ends of each housing 14, a magnetic connection is established.

In some embodiments, and referring now to FIG. 3C, the magnetic connection mechanism comprises magnet release switches 16 located on each housing 14. Magnet release switches 16 are configured such that they shift the female magnetic connection and disconnect the magnets, allowing chin support 30 to be detached from body 10. In some embodiments, the magnet release switches 16 are located on the underside of the housing 14 of body 10. The use of magnets as an attachment mechanism for chin support attachment 30 allows the user to attach and release chin support 30 without direct visuals to guide the chin support to the attachment point. In some embodiments, the magnet release switches 16 are textured to allow the user to locate the switch without needing a direct line of sight, thereby allowing the user to don and doff the collar independently. Examples of textured surfaces may include, without limitations, grooves, bumps, divots, threads, and the like.

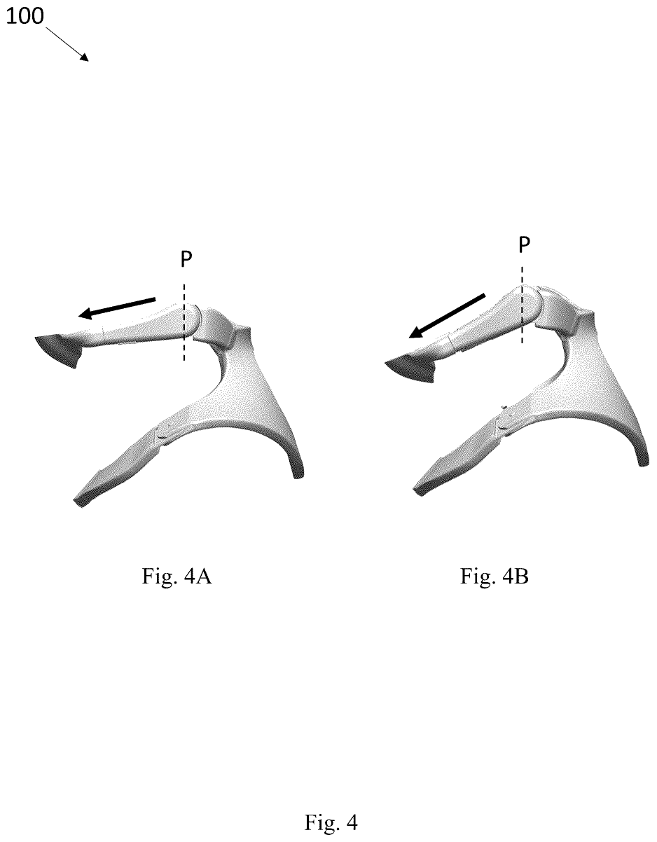

In some embodiments, and referring back to FIG. 3A, housings 14 further comprise a pivot mechanism 17. In some embodiments, pivot connections are located inside housing 14 and allow for a pivotal movement of housing 14, and thereby chin support attachment 30, about a pivot axis P parallel to the length of collar body 10, such that the angle subtended by the collar body and housing 14, and thereby chin support attachment 30, can be adjusted within an angular range. The pivot mechanisms are activated by actuators 18 located on a side of each housing 14, which, when pressed, releases the pivot and allows chin support attachment 30 to be rotated to a desired angle. The user then releases actuators 18 to lock chin support 30 in place. In some embodiments, and referring now to FIG. 4, the chin support attachment 30 can be adjusted at any angle about a pivot axis P parallel to the length of collar body 10. In some embodiments, the angle can be adjusted to angle between 5° and 90°, to at least 5°, at least 10°, at least 15°, at least 20°, at least 25°, at least 30°, at least 40°, at least 50°, at least 60°, at least 70°, at least 80°, or at least 90°. In some embodiments, actuators 18 are textured to allow the user to locate actuators 18 without a direct line of sight. Examples of textured surfaces may include, without limitations, grooves, bumps, divots, threads, and the like.

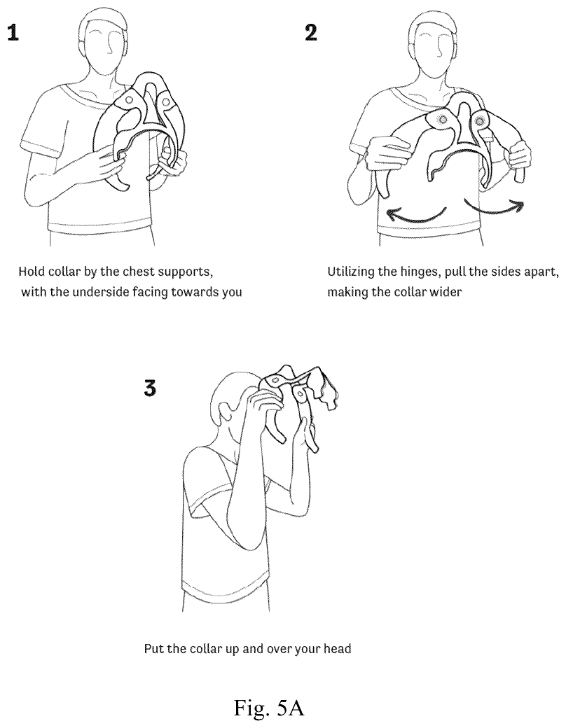

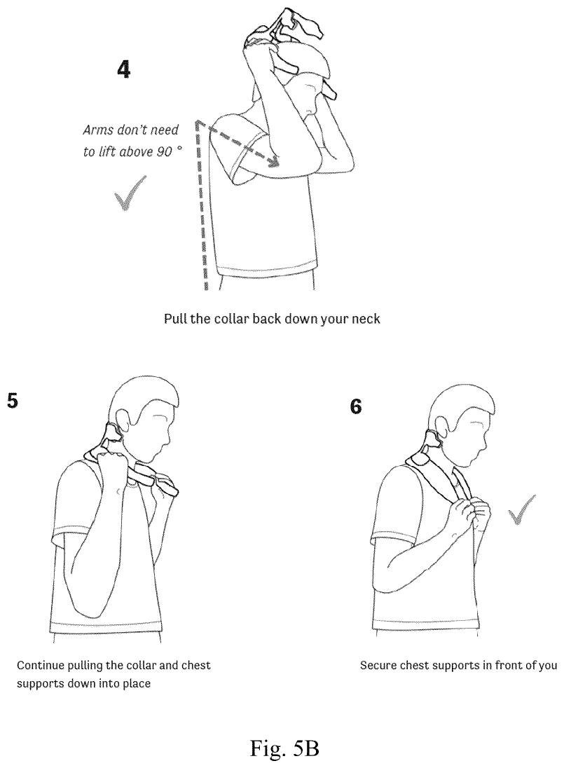

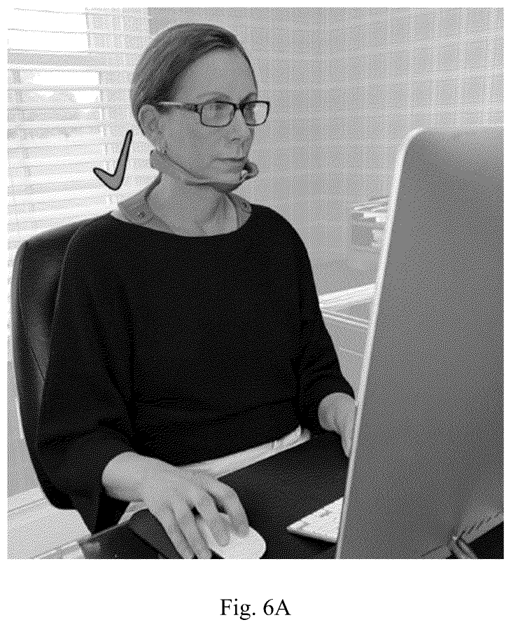

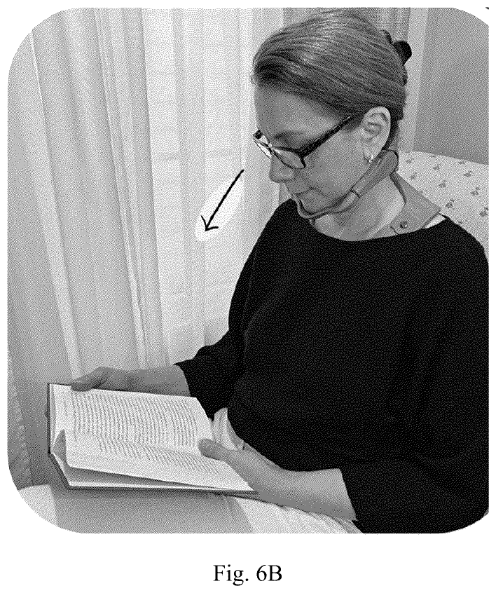

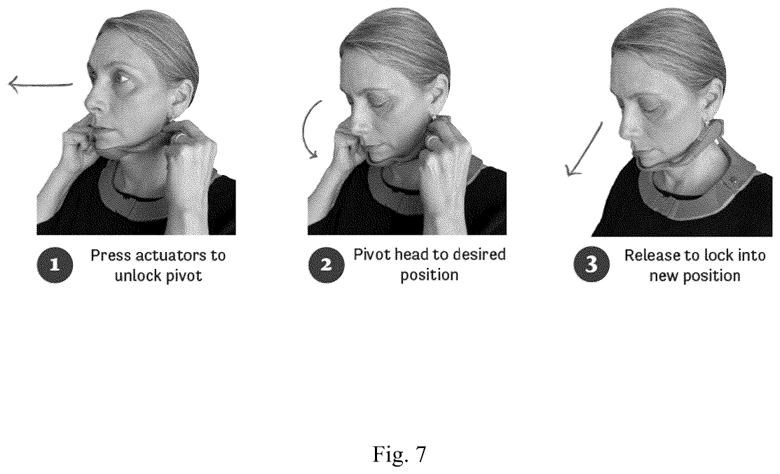

In a preferred embodiment, and referring now to FIG. 5A and FIG. 5B, a user dons collar 100 by holding the collar by the chest supports, with the inner surface of the collar pointing towards the user and utilizing the hinges to pull the sides apart. The user can then put collar 100 up and over their head and back down their neck. Once the collar is in place, the user can then secure the chest supports by buckling the straps. To attach chin support 30, the user simply guides the lateral sides of chin support 30 to the ends of housing 14 to establish the magnetic connection. To doff chin support 30, the user pushes on the magnet release switches 16 to shift the internal magnet thereby disconnecting the magnets and releasing chin support 30. The donning mechanism is advantageous since it does not require the user to lift their arms above their heads and allows users to independently don and doff cervical collar 100.

In some embodiments, the present invention relates to a method of fabricating a custom-fitted cervical neck collar. In some embodiment, the method of fabricating a cervical neck collar comprises the steps of: obtaining measurements of the user via a 3-dimensional scan, producing a computerized model of the cervical neck collar such that it conforms to the user's measurements, 3D printing the cervical neck collar components wherein the components comprise a collar body, a chest support and a chin support attachment, and assembling the components.

In some embodiments, each cervical collar is custom fitted to a user. A 3-dimensional scan of the user's upper back, neck and head is performed to obtain patient anatomy data. The 3-dimensional scan is then used to create a customized computerized model of cervical collar 100. The collar design results in a cervical collar that is adapted to the specific anatomy of the user. The resulting cervical collar 100 may be adjusted to any desired shape, curvature, or size such that it conforms exactly to the user's anatomical data. The custom-fit prevents bruising and pinching of the skin and muscles and distributes the weight of the collar evenly to avoid localized pressure points and ensure comfort. In some embodiments, the computerized model is 3D printed in parts to produce custom-fitted body 10, custom-fitted chest supports 20 and custom-fitted chin support 30.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An adjustable cervical collar, comprising:
a collar body comprising a base configured to rest on shoulders of a user, and a neck extending upward from the base, the neck comprising at least one connection point;
a chest support comprising first and second portions hingedly connected to the collar body, and configured to secure the cervical collar about a neck of the user when connected to one another; and
a chin support attachment configured to detachably connect to the connection point of the collar body.

2. The cervical collar of claim 1, wherein the chin support is configured to detachably connected to the at least one connection point via at least one pivot connection, configured to allow the chin support to rotate about a pivot axis.

3. The cervical collar of claim 1, wherein the at least one connection point comprises left and right connection points, and the chin support is configured to detachably connect to the left and right connection points via left and right pivot connections to allow the chin support to rotate about a pivot axis.

4. The cervical collar of claim 3, wherein the pivot axis is parallel to a transversal axis of the user.

5. The cervical collar of claim 3, wherein the first and second side portions of the collar body comprise an actuator configured to rotate the chin support attachment through a range of motion.

6. The cervical collar of claim 5, further comprising a button configured to allow rotation of the actuator about the pivot axis when pressed, and lock the chin support attachment in position relative to the pivot axis when released.

7. The cervical collar of claim 2, wherein the at least one connection point comprises a guide structure, allowing the user to easily attach the chin support attachment by guiding the chin support attachment into place, without the need for a direct line of sight.

8. The cervical neck collar of claim 2, wherein the collar further comprises a release mechanism configured such that, by pressing on a switch located near the connection point, releases the chin support attachment from the collar body.

9. The cervical neck collar of claim 1, wherein the first and second portions of the chest support are hingedly attached to one another at a chest support attachment point to allow for pivotal movement about a chest pivot axis such that the first and second portion of the chest support can each be rotated independently.

10. The cervical neck collar of claim 9, wherein the chest pivot axis is parallel to the frontal plane of the user.

11. The cervical collar of claim 9, wherein the chest support attachment point further comprises a detent that restricts the angular range of motion of the first and second portions of the chest support relative to one another about the chest pivot axis.

12. The cervical collar of claim 9, wherein the first and second portions of the chest support are configured to be rotated to create a size-adjustable opening, through which the collar can be donned by the user.

13. The cervical neck collar of claim 9, wherein the first and second portions of the chest support are releasably attached at the chest support attachment point via a strap buckle which secures the cervical neck collar to the user.

14. The cervical neck collar of claim 1, wherein the body, chest support and chin support attachment are made of a material selected from plastics, metals, any organic or nonorganic polymers, polylactic acid (PLA), thermoplastic polyurethane (TPU), or composite materials.

15. The cervical neck collar of claim 1, wherein at least one of the collar body, chest support and chin support attachment is asymmetrical and sized or shaped in a way to fit specific anatomy of the user.

16. A method of fabricating a cervical neck collar, comprising the steps of:
obtaining user measurements via a 3-dimensional scan;
producing a computerized model of a cervical neck collar that conforms to the user's measurements;
3D printing a cervical neck collar components, wherein the components comprise a collar body, a shoulder support, and a chin support attachment; and
assembling the components to form the cervical neck collar.
